(19) United States
(12) Patent Application Publication
(10) Pub. No.: US 2004/0057922 A1
(43) Pub. Date: Mar. 25, 2004

(54) Cosmetic and/or pharmaceutical agent

(76) Inventors: Karl Heinz Schmid, Mettmann (DE); Hans-Christian Raths, Monheim (DE); Anke Eggers, Duesseldorf (DE); Ingomar Mrozek, Duesseldorf (DE)

Correspondence Address: COGNIS CORPORATION, PATENT DEPARTMENT, 300 BROOKSIDE AVENUE, AMBLER, PA, 19002, US

(21) Appl. No.: 10/466,513

(22) PCT Filed: Jan. 9, 2002
(86) PCT No.: PCT/EP02/00126

(30) Foreign Application Priority Data
Jan. 18, 2001 (DE) 101 02 007.4

(51) Int. Cl. A61K 7/06; A61K 7/11; A61K 7/075; A61K 7/08

(52) U.S. Cl. 424/70.13; 424/70.22

(57) ABSTRACT

Preparations comprising at least one acylated amino acid in an amount of from 2 to 50% by weight and an alk(en)yl oligoglycoside in an amount of from 50 to 98% by weight, based on the total weight of the combined acylated amino acid and alk(en)yl oligoglycoside, are described. Cosmetic and/or pharmaceutical compositions containing said preparations are also described along with methods of producing foam and stabilizing foam produced by surfactant compositions.

### FIELD OF THE INVENTION

This invention relates to preparations of at least one acylated amino acids and alkyl and/or alkenyl oligoglycosides of defined composition and to their use as foaming agents.

1. Prior Art

Anionic surfactants, such as fatty alcohol ether sulfates, show excellent foaming properties in cosmetic preparations, such as for example hair shampoos and the like, and can easily be adjusted to high viscosities by addition of thickeners, the viscosity level to be reached being dependent inter alia on the thickener used. A very common thickener is, for example, sodium chloride with which a 10% aqueous solution of a C_{12/14} ether sulfate containing 2 mol ethylene oxide can be adjusted to a viscosity of 2,000 mPas (DGF H II-4) with 4% sodium chloride. However, it is also becoming increasingly desirable to be able to use surfactants which do not contain any ethylene oxide because it has been found that unwanted secondary products can be formed during the storage of “ether-sulfate-containing” formulations, particularly in the presence of light. Accordingly, there is a demand on the market for “ether-sulfate-free” preparations.

The problem addressed by the present invention, therefore, was to provide “ether-sulfate-free” surfactant mixtures which would have excellent foaming properties and would be easy to thicken.

2. Description of the Invention

The present invention relates to a cosmetic and/or pharmaceutical preparation containing

(a) 2 to 50% by weight, preferably 5 to 25% by weight and more particularly 7 to 15% by weight of at least one acylated amino acid,

(b) 98 to 50% by weight, preferably 95 to 75% by weight and more particularly 93 to 85% by weight of alkyl and/or alkenyl oligoglycosides,

with the proviso that the quantities shown add up to 100% by weight, optionally with water.

The present invention also relates to the use of the surfactant mixture according to the invention as a foaming composition.

It has surprisingly been found that mixtures containing acylated amino acids and alkyl and/or alkenyl oligoglycosides with a defined composition—in contrast to acylated amino acids and alkyl and/or alkenyl oligoglycosides on their own—have excellent foam properties (inter alia stable foams). Accordingly, the surfactant mixtures according to the invention may also be used as foam regulators in surface-active preparations. In addition, surfactant mixtures such as these are easy to foam. For example, 10% aqueous solutions of the surfactant mixtures according to the invention can be adjusted with sodium chloride to the viscosity range of 2,000 mPas to 5,000 mPas (DGF H II-1) required for shampoos. The surfactant mixtures according to the invention are therefore an advantageous alternative for “ether-sulfate-free” preparations.

Acylated Amino Acids

Acylated amino acids in the context of the invention are any compounds which are obtainable by acylation of amino acids with fatty acid halides corresponding to formula (I):

R¹COX (I)

in which R¹ is an alkyl or alkenyl group containing 6 to 22, preferably 8 to 18 and more particularly 12 to 16 carbon atoms and X is chlorine, bromine or iodine, preferably chlorine, by any of the standard methods known from the prior art. Typical acid halides are octanoyl chloride, nonanoyl chloride, decanoyl chloride, undecanoyl chloride, lauroyl chloride, tridecanoyl chloride, myristyl chloride, palmitoyl chloride, stearoyl chloride, oleoyl chloride and mixtures thereof. The fatty acid halides are used in a molar ratio of acylatable compound to acid halide of 1 to 1.5 and preferably 1.1 to 1.3% by weight for the production of the surfactant mixtures according to the invention. The acylated amino acids thus produced have a degree of acylation of at least 60, preferably 70 and more particularly 85%.

Preferred acylated amino acids are obtained by reaction of glutamic acid, sarcosine, aspartic acid, alanine, valine, leucine, isoleucine, proline, hydroxyproline, lysine, glycine, serine, cystein, cystine, threonine, histidine and salts thereof and, more particularly, glutamic acid, sarcosine, aspartic acid, lysine, glycine and the monosodium salts thereof in optically pure form or as racemic mixtures with fatty acid halides corresponding to formula (I). In one particular embodiment of the invention, cocoacyl glutamate is used.

The acylated amino acids or their salts are used in the surfactant mixture according to the invention in quantities of 2 to 50, preferably 5 to 25 and more particularly 7 to 15% by weight, based on the active substance content of the composition as a whole.

Alkyl and/or Alkenyl Oligoglycosides

Alkyl and/or alkenyl oligoglycosides are known nonionic surfactants which correspond to formula (II):

R²O-[G]ₚ (II)

where R² is an alkyl and/or alkenyl group containing 4 to 22 and preferably 12 to 16 carbon atoms, G is a sugar unit containing 5 or 6 and preferably 6 carbon atoms and p is a number of 1 to 10. They may be obtained by the relevant methods of preparative organic chemistry. The overviews presented by Bierman et al. in Starch/Stärke 45, 281 (1993), by B. Salka in Cosm. Toil. 108, 89 (1993) and by J. Kahre et al. in SÖFW-Journal No. 8, 598 (1995) are cited as representative of the extensive literature available on this subject.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (II) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view. The alkyl or alkenyl radical R² may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R² may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} coconut oil fatty alcohol having a DP of 1 to 3 are preferred.

The alkyl and/or alkenyl oligoglycosides are used in the surfactant mixture according to the invention in quantities of 98 to 50, preferably 95 to 75 and more particularly 93 to 85% by weight, based on the active substance content of the composition as a whole.

Polyols

In one particular embodiment of the invention, 0 to 15, preferably 0.2 to 5 and more particularly 0.5 to 3% by weight of polyols, for example glycerol, ethylene glycol, propylene glycol, dipropylene glycol, 1,3-butylene glyol, butane-1,2-diol, butane-1,4-diol, sorbitol, mannitol, erythritol, pentaerythritol, may be added as an additional component.

Production of Acylated Amino Acids

The acylated amino acids are produced by the methods known from the chemical literature. The reaction may also be carried out using solvents, such as ethanol, isopropanol, propylene glycol, etc.

Commercial Applications

The preparations according to the invention may be diluted with water to any concentration. The water content may be from 10 to 80% by weight and is preferably from 30 to 70% by weight and more particularly from 40 to 60% by weight. They may be used in surface-active preparations in quantities of 0.05 to 40, preferably 0.5 to 25 and more particularly 2.0 to 10% by weight, based on the active substance content.

Surface-active preparations in the context of the invention are, preferably, laundry and dishwashing detergents, cleaners and also cosmetic and/or pharmaceutical preparations, more particularly cosmetic and/or pharmaceutical preparations. These surface-active preparations may contain pearlizing waxes, consistency factors, thickeners, superfatting agents, stabilizers, silicone compounds, fats, waxes, lecithins, phospholipids, antioxidants, deodorants, antiperspirants, antidandruff agents, swelling agents, tyrosine inhibitors, hydrotropes, solubilizers, preservatives, perfume oils, dyes, surfactants and other typical ingredients encountered, for example, in laundry detergents, dishwashing detergents and cleaning compositions as further auxiuliaries and additives. Preferred cosmetic and/or pharmaceutical preparations are oral hygiene and dental care preparations, hair shampoos, hair lotions, foam baths, shower baths, creams, gels, lotions, alcoholic and aqueous/alcoholic solutions and emulsions.

The mixtures according to the invention may advantageously be used as foaming agents or as emulsifiers in the surface-active preparations.

Typical cosmetic and/or pharmaceutical cleansing preparations preferably have the following composition (based on the active substance content):

(a) 0.05 to 15, preferably 0.5 to 10 and more particularly 2.0 to 7.5% by weight of the mixture according to the invention of at least one acylated amino acid with alkyl and/or alkenyl oligoglcosides

(b) 0.05 to 15, preferably 0.5 to 10 and more particularly 2.5 to 7.5% by weight of betaines and optionally

(c) 0 to 15, preferably 0.5 to 10 and more particularly 2.5 to 7.5% by weight of other anionic surfactants,

with the proviso that the quantities shown add up to 100% by weight, optionally with other auxiliaries and additives.

Typical liquid laundry and dishwashing detergents and cleaners preferably have the following composition (based on the active substance content):

(a) 2.0 to 40, preferably 7 to 25 and more particularly 10 to 20% by weight of the mixture according to the invention of at least one acylated amino acid with alkyl and/or alkenyl oligoglcosides

(b) 0.05 to 15, preferably 0.5 to 10 and more particularly 2.5 to 7.5% by weight of betaines and optionally

(c) 2.5 to 30, preferably 7 to 25 and more particularly 10 to 20% by weight of other anionic surfactants,

with the proviso that the quantities shown add up to 100% by weight, optionally with other auxiliaries and additives.

Typical cosmetic and/or pharmaceutical emulsions preferably have the following composition (based on the active substance content):

(a) 0.05 to 15, preferably 0.5 to 10 and more particularly 1 to 5% by weight of the mixture according to the invention of at least one acylated amino acid with alkyl and/or alkenyl oligoglcosides

(b) 3 to 30, preferably 5 to 20 and more particularly 7 to 15% by weight of oil components and optionally

(c) 0.5 to 20 and preferably 2.5 to 10% by weight of consistency factors, with the proviso that the quantities shown add up to 100°/ by weight, optionally with other auxiliaries and additives.

Other Auxiliaries

Waxes

Suitable waxes are inter alia natural waxes such as, for example, candelilla wax, carnauba wax, Japan wax, espartograss wax, cork wax, guaruma wax, rice germ oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs) and correspond to the following general formula:

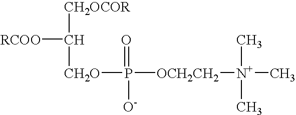

where R typically represents linear aliphatic hydrocarbon radicals containing 15 to 17 carbon atoms and up to 4 cis-double bonds. Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially coconut fatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Antioxidants

Antioxidants which interrupt the photochemical reaction chain that is initiated when UV rays penetrate into the skin may also be added. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic fragrances. Natural fragrances include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosm tische Färbemittel” of the Farbstoffkommission d r Deutsch n Forschungsgemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 80% by weight and is preferably from 5 to 50% by weight and more particularly from 7 to 10% by weight, based on the particular preparation. The preparations may be produced by standard cold or hot emulsification processes and are preferably produced by the phase inversion temperature (PIT) method.

### EXAMPLES

| | |
|---|---|
| A1 | sodium salt of an N-coconut-C₈₋₁₆-acyl glutamate |
| A2 | sodium salt of an N-coconut-C₈₋₁₆-aspartate |
| B1 | C₁₂₋₁₆ alkyl polyglycoside, DP = 1.42 (DP = average degree of |
| | glucosidation |
| B2 | C₁₂₋₁₆ alkyl polyglycoside, DP = 1.53 (DP = average degree of |
| | glucosidation) |
| C | C_{12/14} ether sulfate containing 2 mol EO |

The surfactant mixtures according to the invention (Examples 1 to 4), the comparison surfactant mixture (C3) and the individual components (Comparison Examples C1 and C2) were tested for their foaming capacity in hard water. To this end, the “whipped” volume (ml) was determined at different times by Götte's whipped foam method (DIN 53902, 3/81; 14° dH, pH 6, 40° C.). In addition, 4% NaCl solution was added to determine whether the surfactant mixtures according to the invention (Example 5) could be thickened by comparison with acylated amino acids (C1), alkyl polyglycosides (C2) or ether sulfates (C4) alone. The Brookfield viscosity (DGF H II-4) was determined at 20° C. on an aqueous solution containing 10% by weight surfactant or surfactant mixture according to the invention after the addition of 4% sodium chloride. The results are set out in Tables 1 and 2.

TABLE 1
Foaming capacity (quantities in g/l active substance)

| Composition | 1 | 2 | 3 | 4 | 5 | C1 | C2 | C3 |
|---|---|---|---|---|---|---|---|---|
| A1 | 1.0 | — | 1.0 | 0.5 | 0.2 | 2.0 | — | 1.5 |
| A2 | — | 1.0 | — | 0.5 | — | — | — | — |
| B1 | 1.0 | — | — | — | 1.8 | — | 2.0 | 0.5 |
| B2 | — | 1.0 | 1.0 | 1.0 | — | — | — | — |
| C1 | — | | | | — | — | — | |
| Foam volume | | | | | | | | |
| [ml] | | | | | | | | |
| After 30 s | 450 | 510 | 490 | 500 | 430 | 350 | 250 | 400 |
| After 5 mins. | 440 | 490 | 470 | 480 | 430 | 0 | 240 | 150 |
| After 10 mins. | 430 | 480 | 460 | 470 | 415 | 0 | 235 | 100 |
| After 15 mins. | 430 | 470 | 460 | 460 | 415 | 0 | 230 | 0 |
| After 20 mins. | 430 | 470 | 450 | 460 | 410 | 0 | 220 | 0 |

TABLE 2
Viscosity (quantities in g/10 ml active substance)

| Composition | 5 | C1 | C2 | C4 |
|---|---|---|---|---|
| A1 | 0.2 | 2.0 | — | — |
| B1 | 1.8 | — | 2.0 | — |
| C1 | — | — | — | 2 |
| Viscosity [mPas] | 5600 | 200 | 1250 | 2500 |

1. Cosmetic and/or pharmaceutical preparation containing
(a) 2 to 50% by weight of at least one acylated amino acid and
(b) 98 to 50% by weight of alkyl and/or alkenyl oligoglycosides,
with the proviso that the quantities shown add up to 100% by weight, optionally with water.

2. Preparation as claimed in claim 1, characterized in that
(a) 5 to 25% by weight of at least one acylated amino acid and
(b) 95 to 75% by weight of alkyl and/or alkenyl oligoglycosides,
with the proviso that the quantities shown add up to 100% by weight, optionally with water, are used.

3. Preparation as claimed in claims 1 and/or 2, characterized in that acylated amino acids obtained by reaction of amino acids with fatty acid halides corresponding to formula (I):
R¹COX (I)
in which R¹ is an alkyl or alkenyl group containing 6 to 22 carbon atoms and x is chlorine, bromine or iodine, are used.

4. Preparation as claimed in at least one of claims 1 to 3, characterized in that acylated amino acids with a degree of acylation of at least 70% are used.

5. Preparation as claimed in at least one of claims 1 to 4, characterized in that acylated amino acids formed by reaction of glutamic acid, sarcosine, aspartic acid, alanine, valine, leudine, isoleucine, proline, hydroxyproline, lysine, glysine, serine, cysteine, cystine, threonine, histidine and salts thereof with fatty acid halides corresponding to formula (I) are used.

6. Preparation as claimed in at least one of claims 1 to 5, characterized in that glutamic acid, sarcosine, aspartic acid, lysine, glycine and their monosodium salts are used.

7. Preparation as claimed in at least one of claims 1 to 6, characterized in that alkyl and/or alkenyl glycosides corresponding to formula (II):
R²O-[G]ₚ (II)
in which R² is an alkyl and/or alkenyl group containing 4 to 22 carbon atoms, G is a sugar unit containing 5 or 6 carbon atoms and p is a number of 1 to 10, are used.

8. Preparation as claimed in at least one of claims 1 to 7, characterized in that alkyl and/or alkenyl oligoglycosides corresponding to formula (II), in which R² is an alkyl and/or alkenyl group containing 12 to 16 carbon atoms, G is a sugar unit containing 6 carbon atoms and p is a number of 1 to 10, are used.

9. Cosmetic and/or pharmaceutical compositions containing 0.05 to 40% by weight of the preparation claimed in at least one of claims 1 to 8.

10. The use of the preparation claimed in claim 1 as a foaming agent.
